(19) United States
(12) Patent Application Publication
(10) Pub. No.: US 2024/0173804 A1
(43) Pub. Date: May 30, 2024

(54) METHOD FOR LASER DRILLING SYRINGE BORE TO ELIMINATE TUNGSTEN CONTAMINATION

(71) Applicant: CORNING INCORPORATED, Corning, NY (US)

(72) Inventors: Anatoli Anatolyevich Abramov, Painted Post, NY (US); Connor Thomas O'Malley, Painted Post, NY (US)

(21) Appl. No.: 18/522,807

(22) Filed: Nov. 29, 2023

Related U.S. Application Data
(60) Provisional application No. 63/428,936, filed on Nov. 30, 2022.

(51) Int. Cl. B23K 26/382; A61M 5/31

(52) U.S. Cl. CPC B23K 26/382; A61M 5/3129

(57) ABSTRACT

Provided are embodiments of a method of forming a syringe barrel. In the method, a bore is drilled through a tip of the syringe barrel with a first laser to provide fluid communication with an interior cavity of the syringe barrel. The interior cavity is defined by a tubular wall of the syringe barrel. A surface of the bore is treated with a second laser to remelt the surface of the bore. The tubular wall and tip are made of a glass material. Advantageously, forming the bore of the syringe barrel using laser drilling and treating avoids tungsten contamination.

### CROSS-REFERENCE TO RELATED APPLICATIONS

This application claims the benefit of priority under 35 U.S.C. § 119 of U.S. Provisional Application Ser. No. 63/428,936 filed on Nov. 30, 2022, the content of which is relied upon and incorporated herein by reference in its entirety.

### BACKGROUND OF THE DISCLOSURE

The present disclosure generally relates to glass syringes and in particular to glass syringes without tungsten contamination.

Syringes having a glass barrel are advantageous for use in certain applications, such as where high precision is needed and where a high gas barrier is needed. However, the conventional method by which glass syringe barrels are formed involves the use of a tungsten pin to create the bore in the tip. The use of tungsten in the syringe barrel forming process leads to tungsten contamination, which can cause aggregation and particle formation in protein solutions. Various attempts have been made to address tungsten contamination, including washing steps to try to remove tungsten reside as well as replacing the tungsten tips with other metals or ceramics. Washing does not typically remove all of the residue, leaving the possibility for undesired reactions with the remaining tungsten. Tips made of other metals may lead to different forms of contamination, and tips made of ceramics are too brittle to use for low diameter bores.

### SUMMARY OF THE DISCLOSURE

According to aspect (1), a method is provided. The method comprising: drilling a bore through a tip of a syringe barrel with a first laser to provide fluid communication with an interior cavity of the syringe barrel, the interior cavity being defined by a tubular wall of the syringe barrel: treating a surface of the bore with a second laser to remelt the surface of the bore: wherein the tubular wall and tip comprise a glass material.

According to aspect (2), the method of aspect (1) is provided, wherein the first laser operates at a first wavelength and the second laser operates at a second wavelength and wherein the first wavelength is different from the second wavelength.

According to aspect (3), the method of aspect (2) is provided, wherein the first wavelength is 1200 nm or less.

According to aspect (4), the method of aspect (3) is provided, wherein the first wavelength is in an ultraviolet or visible range.

According to aspect (5), the method of aspect (4) is provided, wherein the first wavelength is 266 nm, 355 nm, or 532 nm.

According to aspect (6), the method of any one of aspects (1)-(5) is provided, wherein drilling the bore further comprises pulsing the first laser in pulses of 25 nanoseconds or less.

According to aspect (7), the method of any one of aspects (2)-(6) is provided, wherein the second laser is a CO2 laser.

According to aspect (8), the method of aspect (7) is provided, wherein the second wavelength is in a range from 9200 nm to 10600 nm.

According to aspect (9), the method of any one of aspects (2)-(6) is provided, wherein the second laser is a CO laser.

According to aspect (10), the method of aspect (9) is provided, wherein the second wavelength is in a range from 5200 nm to 6000 nm.

According to aspect (11), the method of any one of aspects (1)-(10) is provided, wherein treating the surface of the bore comprises pulsing the second laser.

According to aspect (12), the method of any one of aspects (1)-(10) is provided, wherein the second laser is a continuous wave laser.

According to aspect (13), the method of any one of aspects (1)-(12) is provided, wherein drilling the bore further comprises tapering the bore from a first diameter at a first end of the tip to a second diameter at a first depth of the tip, the second diameter being less than the first diameter.

According to aspect (14), the method of aspect (13) is provided, wherein drilling the bore further comprises tapering the bore from the second diameter at a second depth of the tip to a third diameter at a second end of the tip, the third diameter being greater than the second diameter.

According to aspect (15), the method of any one of aspects (1)-(12) is provided, wherein the bore comprises a length and a diameter and wherein a ratio of the length to the diameter is from 15:1 to 20:1.

According to aspect (16), the method of aspect (15) is provided, wherein the diameter is 2 mm or less.

According to aspect (17), the method of aspect (15) or (16) is provided, wherein the length is in a range from 5 mm to 10 mm.

According to aspect (18), the method of any one of aspects (1)-(17) is provided, wherein drilling the bore is performed at room temperature.

According to aspect (19), the method of any one of aspects (1)-(17) is provided, wherein drilling the bore is performed at a temperature at or within 20° C. of an annealing temperature of the glass material.

According to aspect (20), the method of aspect (19) is provided, wherein the temperature is below a softening point of the glass material.

According to aspect (21), the method of any one of aspects (1)-(20) is provided, wherein, prior to drilling the bore, the method further comprises pressing the tubular wall to reduce a diameter of the tubular wall to form the tip.

According to aspect (22), the method of any one of aspects (1)-(21) is provided, wherein drilling the bore further comprises drilling a plurality of other bores of a plurality of other syringe barrels in parallel with the bore of the syringe barrel using a plurality of other first lasers or by splitting a beam of a single first laser.

According to aspect (23), the method of any one of aspects (1)-(22) is provided, wherein treating the surface of the bore further comprises treating a plurality of other surfaces of a plurality of other bores in parallel with the surface of the bore using a plurality of other second lasers or by splitting a beam of a single second laser.

According to aspect (24), a syringe barrel is provided. The syringe barrel comprising: a tubular wall defining an interior cavity: a tip comprising a first end, a second end, and a bore extending from the first end to the second end, the bore being in fluid communication with the interior cavity: wherein the tubular wall and the tip comprise a glass material; and wherein the bore comprises a surface region substantially free of tungsten. According to aspect (25), the syringe barrel of aspect (24) is provided, wherein the bore comprises a length and a diameter and wherein a ratio of the length to the diameter is 15:1 to 20:1.

According to aspect (26), the syringe barrel of aspect (25) is provided, wherein the length is in a range from 5 mm to 10 mm.

According to aspect (27), the syringe barrel of aspect (25) or (26) is provided, wherein the diameter is 2 mm or less.

According to aspect (28), the syringe barrel of aspect (27) is provided, wherein the diameter is in a range from 0.4 mm to 0.8 mm.

According to aspect (29), the syringe barrel of any of aspects (24)-(28) is provided, wherein the bore comprises a first tapered region that decreases in diameter from the first end to a first depth of the tip.

According to aspect (30), the syringe barrel of any of aspects (24)-(29) is provided, wherein the bore comprises a second tapered region that increases in diameter from a second depth of the tip to the second end of the tip.

According to aspect (31), the syringe barrel of any of aspects (24)-(30) is provided, wherein the glass material is an aluminosilicate glass or a borosilicate glass.

According to aspect (32), the syringe barrel of any of aspects (24)-(31) is provided, wherein the syringe barrel is compliant with ISO 11040-4:2015.

According to aspect (33), a method of forming a syringe barrel is provided. The method comprising: pressing a tube of glass material between a first former and a second former to form a tip: drilling a bore through the tip with a first laser, the first laser producing a first beam having a first wavelength; treating a surface of the bore with a second laser, the second laser producing a second beam having a second wavelength, the second wavelength being different from the first wavelength.

According to aspect (34), the method of aspect (33) is provided, wherein the first wavelength is 1200 nm or less.

According to aspect (35), the method of aspect (33) or (34) is provided, wherein the second wavelength is in a range from 5200 nm to 6000 nm or from 9200 nm to 10600 nm.

According to aspect (36), the method of any one of aspects (33)-(35) is provided, wherein drilling comprises pulsing the first laser in pulses of 25 nanoseconds or less.

According to aspect (37), the method of any one of aspects (33)-(36) is provided, wherein treating comprises pulsing the second laser in pulses of 25 nanoseconds or less.

According to aspect (38), the method of any one of aspects (33)-(37) is provided, wherein drilling is performed at a temperature below a softening point of the glass material.

According to aspect (39), the method of any one of aspects (33)-(38) is provided, wherein the bore comprises a length and a diameter and wherein a ratio of the length to the diameter is 15:1 to 20:1.

According to aspect (40), the method of any one of aspects (33)-(39) is provided, wherein, during drilling, the first beam is directed through a beam scanner that changes an angle at which the first beam contacts the tip.

According to aspect (41), the method of any one of aspects (33)-(40) is provided, wherein, during treating, the second beam is directed through a beam scanner that changes an angle at which the second beam contacts the surface of the bore.

According to aspect (42), the method of any one of aspects (33)-(41) is provided, further comprising splitting the first beam during drilling so that multiple bores of multiple tips are drilled in parallel.

According to aspect (43), the method of any one of aspects (33)-(42) is provided, further comprising splitting the second beam during treating so that multiple surfaces of multiplied bores are treated in parallel.

According to aspect (44), the method of any one of aspects (33)-(43) is provided, wherein treating the surface of the bore further comprises remelting the glass material up to a depth of 100 μm.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims.

### BRIEF DESCRIPTION OF THE DRAWINGS

The accompanying drawings are included to provide a further understanding, and are incorporated in and constitute a part of this specification. The drawings illustrate one or more embodiments, and together with the description serve to explain principles and operation of the various embodiments. In the drawings:

FIG. 1 depicts a syringe barrel, according to an exemplary embodiment:

FIG. 2 depicts a detail view of a tip of the syringe barrel shown in FIG. 1, according to an exemplary embodiment:

FIG. 3 depicts flow diagram of a method for forming a syringe barrel, according to an exemplary embodiment: and

FIG. 4 depicts a station for forming bores in multiple syringe barrels in parallel, according to an exemplary embodiment.

### DETAILED DESCRIPTION

Embodiments of the present disclosure relate to a method of laser drilling a bore through the tip of a syringe barrel and to a syringe barrel free of tungsten contamination produced according to the disclosed method. As will be described more fully below, the disclosed method involves using a first laser to laser drill a bore through a tip of a syringe barrel followed by treatment of the bore with a second laser to remove any defects and debris on the surface of the bore. In conventional methods of forming a bore in the tip of a syringe barrel, a glass tube is compressed around a tungsten pin, and the contact between the tungsten and the glass creates contamination that may have an undesirable effect on the contents of the syringe. Because no tungsten components are used in the disclosed forming method, tungsten contamination is avoided. These and other aspects and advantages of the disclosed syringe barrel and method of forming same will be described in greater detail below and in relation to the accompanying figures. These exemplary embodiments are provided by way of illustration, and not by way of limitation.

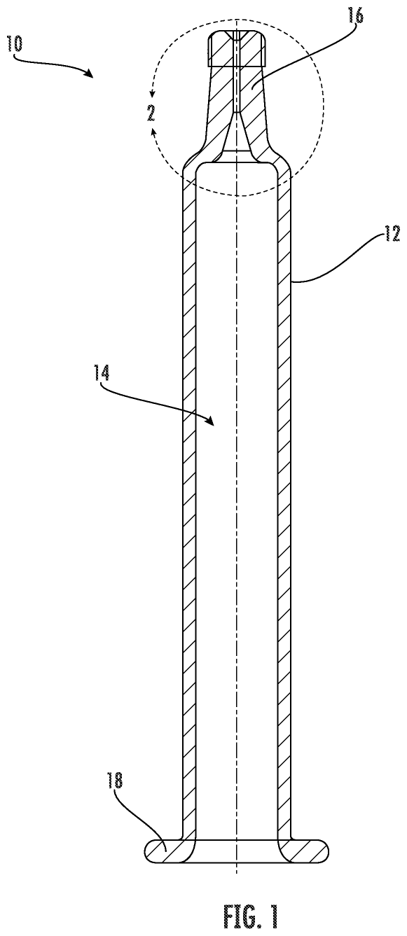

FIG. 1 depicts an embodiment of a syringe barrel 10. The syringe barrel includes a tubular wall 12 defining an interior cavity 14. The syringe barrel 10 has a tip 16 at one end and a flange 18 at the other end. The syringe barrel 10, including the tubular wall 12, the tip 16, and the flange 18, is made of a glass material, such as an aluminosilicate glass. Other glass materials, such as borosilicate glass, are also possible to use for the syringe barrel 10. In one or more embodiments, the syringe barrel 10 may be combined with a needle (not shown) inserted into the tip 16 and bonded in place, and a plunger (not shown) is inserted into the interior cavity 14 from the flange 18 end of the syringe barrel 10 to control dispensing of a fluid contained in the syringe barrel 10.

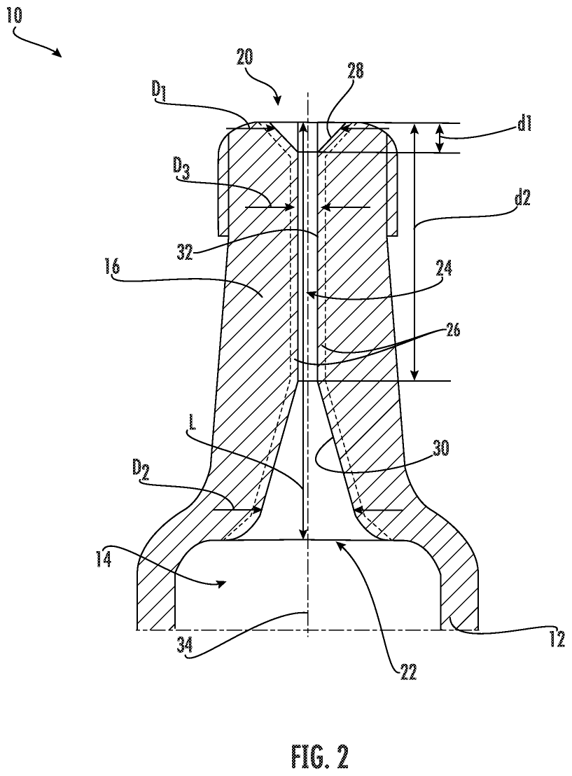

FIG. 2 depicts a detail view of the tip 16 of the syringe barrel 10. The tip 16 has a first end 20, a second end 22, and a bore 24 extending from the first end 20 to the second end 22. The bore 24 is in fluid communication with the interior cavity 14. Further, as will be discussed more fully below, the bore 24 has a surface region 26 substantially free of tungsten. In one or more embodiments, the surface region 26 of the bore 24 includes not only the surface of the bore 24 but also the glass material up to a depth of 5 μm, up to a depth of 10 μm, up to a depth of 20 μm, up to a depth of 30 μm, up to a depth of 50, or up to a depth of 100 μm. In one or more embodiments, the entire tip 16 is substantially free of tungsten, and in one or more embodiments, the entire syringe barrel 10 is substantially free of tungsten. As used herein, “substantially free” means that there is no tungsten contamination on the surface of the bore 24, tip 16, or syringe barrel 10, respectively, and within the glass material of the syringe barrel 10, the glass material contains no more than impurity amounts (e.g., 0.5 mol % or less, 0.05 mol % or less, or 0.005 mol % or less) of tungsten, if any at all.

In one or more embodiments, the bore 24 includes a first tapered region 28 that decreases in a first diameter D1 from the first end 20 to a first depth d1 of the tip 16. In one or more embodiments, the bore 24 includes a second tapered region 30 that increases in a second diameter D2 from a second depth d2 of the tip to the second end 22 of the tip 16. In one or more embodiments, the bore 24 has a central region 32 between the first tapered region 28 and the second tapered region 30 that extends from the first depth d1 to the second depth d2. The central region 32 of the bore 24 has a third diameter D3 that is substantially constant.

In one or more embodiments, the first tapered region 28 has a surface that forms a first angle of up to 15°, up to 30°, or of up to 45° with respect to a longitudinal axis 34 of the syringe barrel 10. In one or more embodiments, the second tapered region 30 has a surface that forms a second angle of up to 15°, up to 30°, or of up to 45° with respect to the longitudinal axis 34 of the syringe barrel 10. In one or more embodiments, the first angle of the first tapered region 28 is the same as the second angle of the second tapered region 30. In one or more embodiments, including the embodiment depicted in FIG. 2, the first angle of the first tapered region 28 is different from the second angle of the second tapered region 30.

In one or more embodiments, the syringe barrel 10 is compliant with ISO 11040-4:2015. This standard establishes an overall length of the syringe barrel 10, thickness of the tubular wall 12, length of the tubular wall 12, outer diameter of the tubular wall 12, and inner diameter of the interior cavity 14 based on the nominal volume of the syringe barrel 10. A length L and the third diameter D3 of the bore 24 can be set by customer specifications. In one or more embodiments, a ratio of the length L to the third diameter D3 is 15:1 to 20:1. In one or more embodiments, the length L is in a range from 5 mm to 10 mm. In one or more embodiments, the third diameter D3 is 2 mm or less, in particular in a range from 0.4 mm to 0.8 mm. In one or more embodiments, the third diameter D3 has a precision of +0.050 mm.

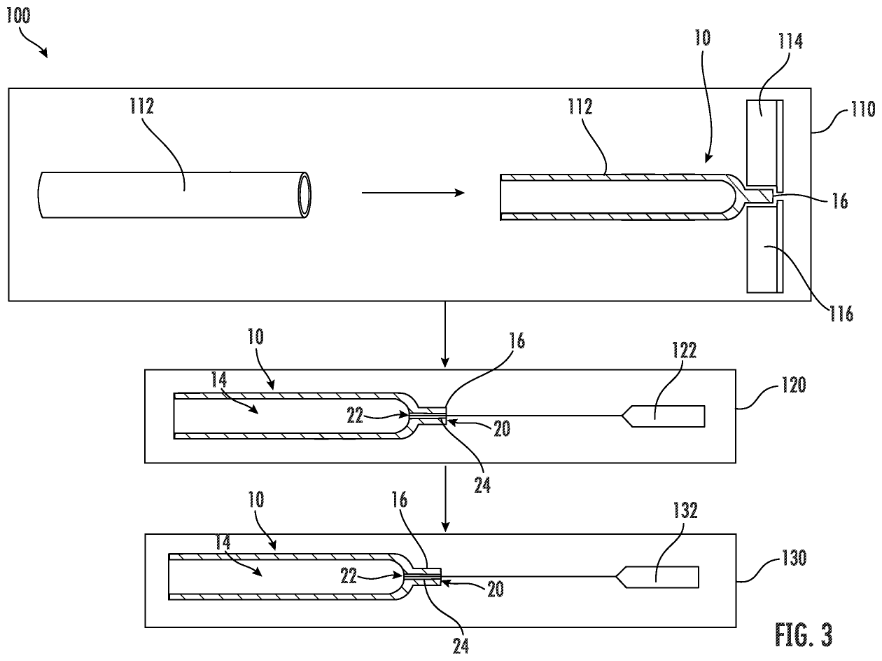

FIG. 3 depicts a flow diagram of a method 100 for forming the syringe barrel 10 such that the bore 24 is substantially free of tungsten contamination. In a first step 110 of the method 100, an end of a tube 112 of glass is pressed between a first former 114 and a second former 116 to reduce a diameter of the tube 112 to form the tip 16, and the remainder of the tube 112 forms the tubular wall 12 of the syringe barrel 10. In one or more embodiments, the tube 112 of glass material is continuously extruded and cut into sections for forming between the formers 114, 116.

In a conventional syringe forming method, a tungsten pin is inserted in the tip at the same time as the formers press onto the outer surface of the tube of glass, which forms the bore. However, the contact between the tungsten and glass leads to tungsten contamination of the glass after the tungsten pin is removed. According to the present disclosure, the bore 24 is not formed at the same time as the tip 16.

Instead, in a second step 120, the bore 24 is drilled through the tip 16 of the syringe barrel 10 with a first laser 122 to provide fluid communication with the interior cavity 14 of the syringe barrel 10. In one or more embodiments, the bore 24 is drilled at room temperature. In one or more other embodiments, the bore 24 is drilled at an elevated temperature. In one or more embodiments, the elevated temperature is below a softening point of the glass material. In one or more embodiments, drilling the bore 24 is performed at a temperature at or within 20° ° C. of an annealing temperature of the glass material. Further, while the term “drilling” is used herein to describe the process of forming a bore 24 with a first laser 122, the particular mechanism of removal may be more accurately described as ablation. As material is ablated from the bore 24 being formed, the focal point of the first laser 122 is scanned and translates deeper into the tip 16.

In one or more embodiments, the first laser 122 operates at a first wavelength. In one or more embodiments, the first wavelength is 1200 nm or less. In one or more embodiments, the first wavelength is in an ultraviolet or visible range. In one or more embodiments, the first wavelength is about 266 nm, about 355 nm, or about 532 nm.

In one or more embodiments, the first laser 122 is pulsed. In one or more embodiments, the first laser 122 is pulsed with pulses of 25 nanoseconds or less, preferably 1 nanosecond or less. In one or more embodiments, the first laser 122 is a continuous wave laser.

As mentioned above, the bore 24 may be tapered at one or both ends of the tip 16. Thus, in one or more embodiments, the bore 24 is drilled such that the bore 24 tapers starting at the first end 20 of the tip 16 and/or at the second end 22 of the tip 16. In one or more embodiments, the tapering of the tip 16 is accomplished by angling the first laser 122 relative to the syringe barrel 10. In one or more embodiments, the tapering of the tip 16 is accomplished by interposing a beam scanner between the first laser 122 and the syringe barrel 10 such that the beam scanner changes the angle at which the laser beam contacts the tip 16. Further, while FIG. 3 depicts the first laser 122 disposed on the first end 20 of the tip 16, the first laser 122 could instead be positioned such that the beam from the first laser 122 initially contacts the second end 22 of the tip 16. Additionally, two first lasers 122 could be used to drill the bore 24 from each end 20, 22 of the tip 16.

In a third step 130 of the method 100, the surface of the bore 24 is treated with a second laser 132 to remelt the surface region of the bore 24. In particular, the beam from the second laser 132 is scanned over the surface region of the bore 24. In this way, glass defects and debris are removed from the laser-drilled bore 24. The treating with the second laser 132 may treat up to a depth of 5 μm, up to a depth of 10 μm, up to a depth of 20 μm, up to a depth of 30 μm, up to a depth of 50, or up to a depth of 100 μm. In one or more embodiments, the second laser 132 operates at a second wavelength. In one or more embodiments, the second wavelength is different from the first wavelength. In one or more embodiments, the second laser has a second wavelength in a range from 9200 nm to 10600 nm. In one or more embodiments, the second laser is a CO2 laser. In one or more embodiments, the second laser has a second wavelength in a range from 5200 nm to 6000 nm. In one or more embodiments, the second laser is a CO laser.

In one or more embodiments, the second laser 132 is pulsed. In one or more embodiments, the second laser 132 is pulsed with pulses of 25 nanoseconds or less, preferably 1 nanosecond or less. In one or more embodiments, the second laser is a continuous wave laser.

In one or more embodiments, the second laser 132 is angled relative to the syringe barrel 10 to treat tapered regions of the bore 24. In one or more embodiments, a beam scanner is interposed between the second laser 132 and the syringe barrel 10 such that the beam scanner changes the angle at which the laser beam of the second laser 132 contacts the surface region of the bore 24. Further, while FIG. 3 depicts the second laser 132 disposed on the first end 20 of the tip 16, the second laser 132 could instead be positioned such that the second laser 132 treats the surface region of the bore 24 from the second end 22 of the tip 16. Additionally, two second lasers 132 could be used to treat the surface region of the bore 24 from each end 20, 22 of the tip 16.

In one or more embodiments, the bore 24 for each syringe barrel 10 can be drilled in 20 seconds or less, in particular 5 seconds or less. Conventional syringe forming techniques allow for a bore to be formed in a syringe tip at the same time as the tip is formed. Throughput in such processes can be, e.g., about 50 syringe barrels per minute. However, as discussed above, such syringe barrels have tungsten contamination, and to decrease tungsten contamination, additional washing steps are required, slowing the syringe barrel forming process. Additionally, the tungsten tips erode quickly and must be replaced periodically, such as every couple of hours of operation. Alternatively, using a ceramic tip to form the bore is limited in terms of the size of the diameter of the bore (>1 mm) because a small diameter ceramic tip is susceptible to breaking.

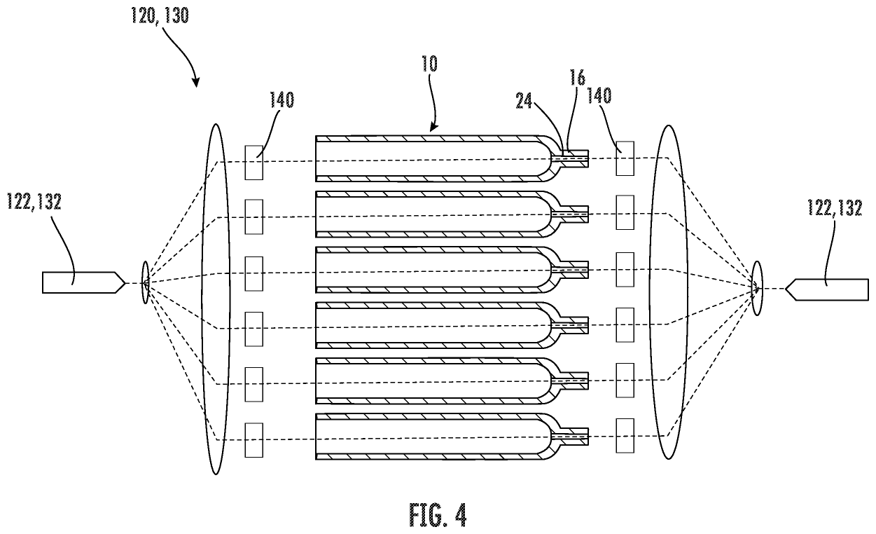

Notwithstanding, to increase throughput according to one or more embodiments, the bores 24 of several tips 16 of syringe barrels 10 can be laser drilled in parallel as shown in FIG. 4. In one or more such embodiments, a plurality of first lasers 122 are used and/or the beam of light from a single first laser 122 is split so that multiple bores 24 can be drilled in parallel. In the embodiment shown in FIG. 4, a single first laser beam 122 is used, and the beam from the first laser beam 122 is split to drill multiple bores 24. Further, in one or more embodiments, treating with the second laser 132 is also performed in parallel using a plurality of second lasers 132 and/or by splitting the beam from a single second laser 132. In the embodiment shown in FIG. 4, a single second laser 132 is used, and the beam from the second laser beam 132 is split to treat multiple bores 24. Further, as discussed above, the first laser 122 or first lasers 122 may be positioned on each end of the syringe barrel 10, and likewise, the second laser 132 or second lasers 132 may be positioned on each end of the syringe barrel 10. Further, as shown in FIG. 4, each beam can be directed through a beam scanner 140 to control the location of the beam focal point, e.g., to provide tapering of the bore 24 or to treat a tapered surface of the bore 24.

Further, in particular for non-tapered bores 24 or bores 24 tapered at only one end, the first laser 122 and the second laser 132 can be arranged on opposite sides of the bore 24. In this way, the first laser 122 can drill the bore 24 from one side, and the second laser 132 can treat the bore 24 from the opposite side. In this way, the syringe barrel 10 or syringe barrels 10 do not need to travel to multiple stations to form the bore 24. Further, treatment with the second laser 132 can take place faster than if the syringe barrels 10 had to travel to a different station.

While not depicted in FIG. 3, the flange 18 (as shown in FIG. 1) can be formed by heating the end of the syringe barrel 10 and pressing the heated end of the syringe barrel 10 against a former.

Syringe barrels 10 produced according to the present disclosure are substantially free, or even entirely free, of tungsten contamination because no tungsten is introduced through the process of forming a bore 24 in the tip 16 as occurs in conventional processes.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more than one component or element, and is not intended to be construed as meaning only one.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

What is claimed is:

1. A method, comprising:
drilling a bore through a tip of a syringe barrel with a first laser to provide fluid communication with an interior cavity of the syringe barrel, the interior cavity being defined by a tubular wall of the syringe barrel;
treating a surface of the bore with a second laser to remelt the surface of the bore;
wherein the tubular wall and tip comprise a glass material.

2. The method of claim 1, wherein the first laser operates at a first wavelength and the second laser operates at a second wavelength and wherein the first wavelength is different from the second wavelength.

3. The method of claim 2, wherein the first wavelength is 1200 nm or less.

4. The method of claim 3, wherein the first wavelength is in an ultraviolet or visible range.

5. The method of claim 4, wherein the first wavelength is 266 nm, 355 nm, or 532 nm.

6. The method of claim 1, wherein drilling the bore further comprises pulsing the first laser in pulses of 25 nanoseconds or less.

7. The method of claim 2, wherein the second laser is a CO2 laser.

8. The method of claim 7, wherein the second wavelength is in a range from 9200 nm to 10600 nm.

9. The method of claim 6, wherein the second laser is a CO laser.

10. The method of claim 9, wherein the second wavelength is in a range from 5200 nm to 6000 nm.

11. The method of claim 1, wherein treating the surface of the bore comprises pulsing the second laser.

12. The method of claim 1, wherein the second laser is a continuous wave laser.

13. The method of claim 12, wherein drilling the bore further comprises tapering the bore from a first diameter at a first end of the tip to a second diameter at a first depth of the tip, the second diameter being less than the first diameter.

14. The method of claim 13, wherein drilling the bore further comprises tapering the bore from the second diameter at a second depth of the tip to a third diameter at a second end of the tip, the third diameter being greater than the second diameter.

15. The method of claim 1, wherein the bore comprises a length and a diameter and wherein a ratio of the length to the diameter is from 15:1 to 20:1.

16. The method of claim 15, wherein the diameter is 2 mm or less.

17. The method of claim 15, wherein the length is in a range from 5 mm to 10 mm.

18. The method of claim 1, wherein drilling the bore is performed at room temperature.

19. The method of claim 1, wherein drilling the bore is performed at a temperature at or within 20° ° C. of an annealing temperature of the glass material.

20. The method of claim 19, wherein the temperature is below a softening point of the glass material.

21. The method of claim 1, wherein, prior to drilling the bore, the method further comprises pressing the tubular wall to reduce a diameter of the tubular wall to form the tip.

22. The method of claim 1, wherein drilling the bore further comprises drilling a plurality of other bores of a plurality of other syringe barrels in parallel with the bore of the syringe barrel using a plurality of other first lasers or by splitting a beam of a single first laser.

23. The method of claim 22, wherein treating the surface of the bore further comprises treating a plurality of other surfaces of a plurality of other bores in parallel with the surface of the bore using a plurality of other second lasers or by splitting a beam of a single second laser.

24. A syringe barrel, comprising:
a tubular wall defining an interior cavity;
a tip comprising a first end, a second end, and a bore extending from the first end to the second end, the bore being in fluid communication with the interior cavity;
wherein the tubular wall and the tip comprise a glass material; and
wherein the bore comprises a surface region substantially free of tungsten.

25. The syringe barrel of claim 24, wherein the bore comprises a length and a diameter and wherein a ratio of the length to the diameter is 15:1 to 20:1.

26. The syringe barrel of claim 25, wherein the length is in a range from 5 mm to 10 mm.

27. The syringe barrel of claim 25, wherein the diameter is 2 mm or less.

28. The syringe barrel of claim 27, wherein the diameter is in a range from 0.4 mm to 0.8 mm.

29. The syringe barrel of claim 24, wherein the bore comprises a first tapered region that decreases in diameter from the first end to a first depth of the tip.

30. The syringe barrel of claim 29, wherein the bore comprises a second tapered region that increases in diameter from a second depth of the tip to the second end of the tip.

31. The syringe barrel of claim 24, wherein the glass material is an aluminosilicate glass or a borosilicate glass.

32. The syringe barrel of claim 24, wherein the syringe barrel is compliant with ISO 11040-4:2015.

33. A method of forming a syringe barrel, comprising:
pressing a tube of glass material between a first former and a second former to form a tip;
drilling a bore through the tip with a first laser, the first laser producing a first beam having a first wavelength;
treating a surface of the bore with a second laser, the second laser producing a second beam having a second wavelength, the second wavelength being different from the first wavelength.

34. The method of claim 33, wherein the first wavelength is 1200 nm or less.

35. The method of claim 34, wherein the second wavelength is in a range from 5200 nm to 6000 nm or from 9200 nm to 10600 nm.

36. The method of claim 35, wherein drilling comprises pulsing the first laser in pulses of 25 nanoseconds or less.

37. The method of claim 33, wherein treating comprises pulsing the second laser in pulses of 25 nanoseconds or less.

38. The method of claim 33, wherein drilling is performed at a temperature below a softening point of the glass material.

39. The method of claim 38, wherein the bore comprises a length and a diameter and wherein a ratio of the length to the diameter is 15:1 to 20:1.

40. The method of claim 39, wherein, during drilling, the first beam is directed through a beam scanner that changes an angle at which the first beam contacts the tip.

41. The method of claim 33, wherein, during treating, the second beam is directed through a beam scanner that changes an angle at which the second beam contacts the surface of the bore.

42. The method of claim 41, further comprising splitting the first beam during drilling so that multiple bores of multiple tips are drilled in parallel.

43. The method of claim 42, further comprising splitting the second beam during treating so that multiple surfaces of multipled bores are treated in parallel.

44. The method of claim 33, wherein treating the surface of the bore further comprises remelting the glass material up to a depth of 100 μm.
